(19) United States
(12) Patent Application Publication
(10) Pub. No.: US 2006/0015128 A1
(43) Pub. Date: Jan. 19, 2006

(54) Surgical devices and method for skin removal

(76) Inventor: Mike Fard, Charlottesville, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/133,239

(22) Filed: May 20, 2005

Related U.S. Application Data
(60) Provisional application No. 60/587,055, filed on Jul. 13, 2004.

(51) Int. Cl. A61B 17/32

(52) U.S. Cl. 606/167

(57) ABSTRACT

Provided are devices and a method for removing skin while causing minimal damage to underlying vascular tissue. An epithelium cutter for removing skin has a handle with a V-shaped blade disposed at one end of the handle. Inner surfaces of the V-shaped blade are sharp, and face away from the handle. In operation, the skin is pulled upwards with tweezers so that it forms a mound, and the V-shaped blade is disposed around the skin mound. Skin removal is initiated by pushing the sharp inner surfaces of the V-shaped blade into the skin mound. The epithelium cutter can remove an arbitrarily long strip of skin. Optionally, to make skin removal easier, the skin can be pre-cut with a rake cutter having 2, 3 or more parallel blades. In this case, a distance between the parallel blades is selected to match a cutting width of the V-shaped blade.

### CROSS-REFERENCE TO RELATED APPLICATIONS

The present application claims the benefit of priority from co-pending provisional application 60/587,055, filed on Jul. 13, 2004, and the complete contents thereof is herein incorporated by reference.

### FIELD OF THE INVENTION

The present invention relates generally to surgical cutting devices and scalpels. More particularly, the present invention relates to a cutting device for removing a strip of skin while minimizing damage to underlying vascular or nerve tissue.

### BACKGROUND OF THE INVENTION

Certain surgical operations require the removal of a patch or strip of a top layer of skin (the epithelium). For example, breast reduction surgery, skin grafts, and certain cosmetic or reconstruction surgical procedures require epithelium removal.

Conventionally, scalpel or scissors are commonly used to remove the epithelium layer. The scalpel or scissors are typically held at a shallow angle, separating the skin from the underlying tissue by cutting. A problem with this conventional technique is that the depth of cut is difficult to control by the surgeon. Often when using a scalpel or scissors to remove epithelium, the blade has a tendency to dig into vascular tissue, causing unnecessary tissue or nerve damage. When removing epithelium, the depth of cut should be accurate and controllable to ensure adequate removal of the epithelium and simultaneously avoid damage to deeper vascular tissue or nerve endings.

Due to these difficulties, proper epithelium removal tends to be time-consuming and difficult for the surgeon and risky for the patient. Damage to vascular tissue increases the risk of complications.

It would be an advance in the art of surgery to provide a simple and effective device and method for epithelium removal causing minimal damage to underlying tissue, and providing a constant and easily controllable depth of cut. It would also be beneficial to have devices and new methods which make epithelium removal faster and easier for the surgeon. Such a device and method could be widely applicable in many surgical procedures.

### SUMMARY OF THE INVENTION

An exemplary embodiment of the invention is to provide a cutting tool with a cutting edge designed to collect and cut epithelium tissue which has been lifted using tweezers or similar devices. Preferably the cutting area is in the shape of V, and the edges of the cutting area are sharpened so as to cut through the epithelium. The device allows removing strips of epithelium simply by applying a slight pushing pressure in the direction of the uplifted section of skin.

Another exemplary embodiment of the invention is to provide a kit which includes both a scoring tool and a cutting tool, wherein the scoring tool provides lines of cut epithelium tissue which can be lifted with tweezers or similar devices, and wherein the cutting tool can cut these lines of epithelium from the surface of the patient's skin simply by pushing a blade along the edge of the skin.

The present invention includes an epithelium cutting device for removing epithelium. The epithelium cutter has a handle for grasping, and a V-shaped blade attached to one end of the handle. The V-shaped blade has sharp inner edges that face away from the handle. Cutting action is provided by moving the handle and blade in a forward direction.

The handle may have a curved shape to accommodate a surgeon's fingers when the handle is placed against a relatively flat surface (e.g. a patients skin surface). The curved shape extends in a vertical direction. The V-shape blade is disposed in a substantially horizontal plane.

The handle may include a flat head portion, also disposed in the horizontal plane, parallel with the V-shaped blade.

The V-shaped blade preferably may have upwardly angled end portions. The end portions may each have an outside surface that is not sharp, thereby avoiding a digging action that might otherwise accompany a generally forward movement of the blade.

The V-shaped blade may comprise two separately fabricated (i.e. sharpened) blades that are butted together (unattached). Alternatively, the separate blades may be attached by welding or soldering, for example, or, the V-shaped blade can be molded, or punched from a single piece of metal, or otherwise formed such that the ends of the V-shape are integral with one another.

The V-shaped blade may be attached to the handle by set screws.

The present invention also includes a rake cutter for creating multiple, parallel cuts in the skin (these being akin to scoring lines). The parallel cuts prepare the skin for removal by the epithelium cutter. The rake cutter preferably comprises a handle for grasping and a plurality of spaced apart, parallel blades attached to one end of the handle. A sheath is disposed on the handle. The sheath can be moved along the handle to cover and uncover varying lengths of the parallel blades so that a depth of cut can be controlled. In addition, the sheath might selectively cover the entire length of the blade when the rake cutter is being stored or shipped.

In use, the uncovered length of the blades is preferably equal among the blades. Also, the sheath preferably has beveled surfaces. The sheath preferably can be locked in position, to fix the length of the exposed blades.

The present invention also encompasses a kit for epithelium removal which includes both the epithelium cutter and the rake cutter. The epithelium cutter preferably has a cutting width (i.e. the width of skin that can be removed by the epithelium cutter in one pass) that is substantially equal to (although being slightly larger (more preferred) or slightly smaller are also acceptable) the spacing between adjacent blades of the rake cutter. This allows the epithelium cutter to completely remove a strip of skin cut by the rake cutter. For example, the spacing between adjacent blades of the rake cutter can be 0-20% more or less than the cutting width of the epithelium cutter.

The present invention also includes a method for skin or epithelium removal. The method includes the steps of cutting multiple parallel cuts with the rake cutter, thereby defining at least one skin strip, and then lifting one end of a skin strip. Then, the V-shaped blade of the epithelium cutter is moved under the skin strip, between the skin and underlying vascular tissue. The epithelium cutter cuts away the skin strip from the vascular tissue, with minimal damage to the vascular tissue. However, due to the ease of use of the epithelium cutter, it is also possible to practice the invention simply by lifting a portion of the skin and cutting the epithelium with the epithelium cutter.

### DESCRIPTION OF THE DRAWING FIGURES

FIG. 1 shows a perspective view of the epithelium cutter device of the present invention.

FIG. 2 shows a side view of the V-shaped blade of the epithelium cutter.

FIG. 3 shows a top view of the V-shaped blade.

FIGS. 4 and 5 show cross sectional views of two different embodiments of the V-shaped blade.

FIG. 6 shows a perspective view of the V-shaped blade.

FIG. 7 shows the epithelium cutter in operation beginning an incision.

FIG. 8 shows a side view of the epithelium cutter separating a strip of skin from underlying tissue (vascular tissue).

FIGS. 9A-9C illustrate alternative embodiments of the V-shaped blade.

FIG. 10 shows a perspective view of a rake cutter for making multiple, parallel cuts.

FIG. 11 shows a top view of the present rake cutter.

FIG. 12 shows a side view of the rake cutter in operation.

FIG. 13 shows a top view of the epithelium cutter removing strips of skin between parallel cuts made by the rake cutter.

FIG. 14 shows a flow diagram describing the method of the present invention.

### DETAILED DESCRIPTION OF THE PREFERRED EMBODIMENTS OF THE INVENTION

The present invention provides devices and a method for removing epithelium in an easily controlled manner that minimizes damage to underlying tissue. The present epithelium cutter does not dig into vascular tissue, and reduces the time required for epithelium removal. The present epithelium cutter and method reduce surgery time, reduce surgeon fatigue, and improve patient safety and surgical outcome.

The present epithelium cutter has a handle and a V-shaped blade, with sharpened inner edges. The ends of the V-shaped blade are preferably angled upwardly, away from the skin when cutting. In operation, the skin is grasped with forceps or tweezers, and pulled upwardly, thereby creating a skin mound. The V-shaped blade is then positioned around the skin mound, and the skin mound is cut away by pushing the V-shaped blade into the skin. Continued pulling by the tweezers and cutting by the V-shaped blade removes a strip of skin, with a controlled depth of cut, and minimal damage to underlying vascular tissue and nerve endings.

Optionally, the skin can be pre-cut with a rake cutter according to the present invention. The rake cutter comprises 2, 3 or more parallel scalpel blades. When the skin is pre-cut with the rake, the V-shaped blade of the epithelium cutter does not need to cut through the skin; the epithelium cutter only separates the skin from the underlying tissue. Cuts made by the rake cutter improve the performance of the epithelium cutter.

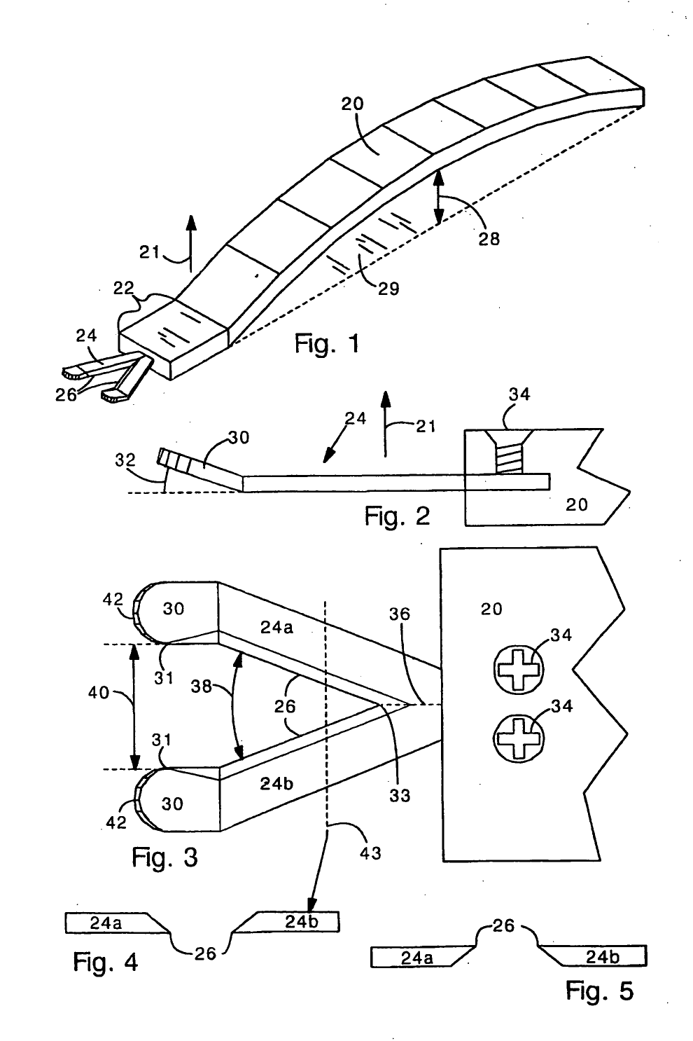

FIG. 1 shows the epithelium cutter according to the present invention. The epithelium cutter has a handle 20 for grasping, and a V-shaped blade 24. The handle 20 is preferably curved in an upward, vertical direction 21 as illustrated. The upward curve in the handle provides a space 28 (e.g., for a surgeons fingers) when the cutter is disposed on a flat surface 29 (e.g., the surface of a patient's skin). The V-shaped blade 24 is disposed in a horizontal plane, orthogonal to the vertical direction 21. The handle 20 may include an optional head portion 22 that is flat and generally planar or parallel with the V-shaped blade 24. The V-shaped blade 24 has sharp inner edges 26 for cutting. The V-shaped blade is attached to the handle so that the sharp edges 26 face away from the handle (i.e., such that the base of the V-shape is attached to the handle 20).

The handle 20 and head portion 22 can be injection molded and made of plastic or made of stainless steel, for example. The handle 20 and head portion 22 can be monolithic. The V-shaped blade 24 can be made of surgical steel, carbide, ceramics, or any other material used for surgical cutting devices.

FIG. 2 shows a close-up side view of the V-shaped blade 24. Preferably, end portions 30 of the V-shaped blade 24 are angled upwardly (i.e., in the vertical direction 21). The angle 32 of the end portions 30 can be in the range of about 0-30 degrees, or 5-15 degrees, for example. The upward angle 32 tends to prevent the V-shaped blade 24 from digging or cutting into vascular tissue, and makes the cutting action more stable and controllable.

The V-shaped blade 24 may be bonded to the handle 20 by molding the handle 20 around the blade 24 (e.g., in an insert molding process). Alternatively, a set screw 34 or adhesive can be used to attach the V-shaped blade 24 to the handle 20.

FIG. 3 shows a close-up top view of the V-shaped blade 24. In one embodiment, the V-shaped blade 24 comprises two blades 24a 24b butted together at boundary 36. The two blades 24a 24b may be welded or soldered together at the boundary 36, or may be unbonded, and held in position by the handle 20. Fabricating the V-shaped blade 24 from two separate blades 24a 24b allows each blade 24a 24b to be machined and sharpened individually, which is helpful for achieving sharp cutting edges close to the corner 33 of the V-shaped blade 24.

The sharp inner edges 26 define an inner angle 38. The inner angle 38 can be in the range of about 10-60 degrees, or, more typically, in the range of 20-30 degrees. The inner angle 38 can be selected to be wider or narrower depending on the type of tissue to be cut, the preference of the surgeon, or depending on other reasons. Typically, the inner angle 38 is fixed in an individual device, but it may be adjustable.

The V-shaped blade 24 has a cutting width 40. The cutting width is the width of the sharp edges 26 as seen in the forward direction (in the plane of the page). The cutting width will typically be in the range of about ⅛-1 inch, or, more typically, in the range of about ¼-½ inch.

Typically, the sharp edges 26 terminate at point 31, on the inside surface of the upwardly angled end portions 30, as shown. In this case, the outside surfaces 42 of the end portions 30 are smooth and rounded, and are not cutting surfaces. Alternatively, portions of the outside surfaces 42 are sharpened.

FIG. 4 shows a cross sectional view of the V-shaped blade 24, cut across line 43. Preferably, the sharp edges 26 are on the bottom of the blade 24 as illustrated.

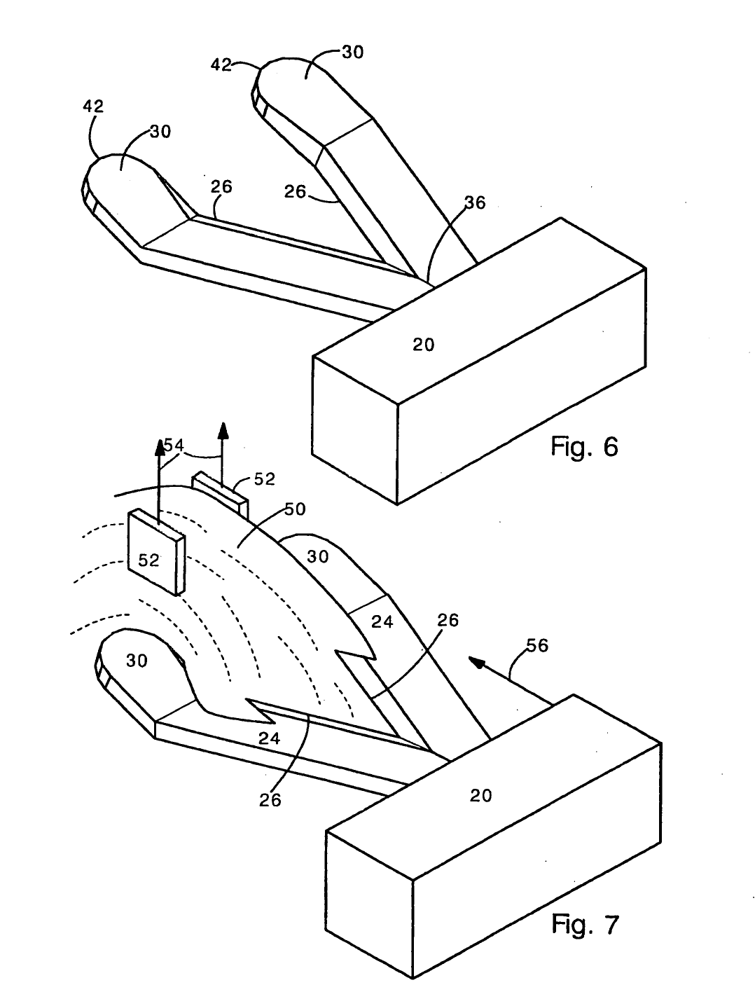

FIG. 5 shows a cross sectional view of an alternative embodiment in which the sharp edges 26 are disposed on a top surface of the V-shaped blade.

Alternatively, the sharp edges 26 can be located between the top and bottom surfaces of the blades 24.

FIG. 6 shows a close-up perspective view of the V-shaped blade 24.

FIG. 7 illustrates the epithelium cutter beginning an incision to remove skin 50. to begin the incision, tweezer or forceps tips 52 are used to grasp the skin 50. The tips are pulled upwardly 54 and lift the skin 50, forming a mound. The V-shaped blade 24 is disposed around the skin mound, and pushed in a forward direction 56 so that the sharp edges 26 cut into the skin 50.

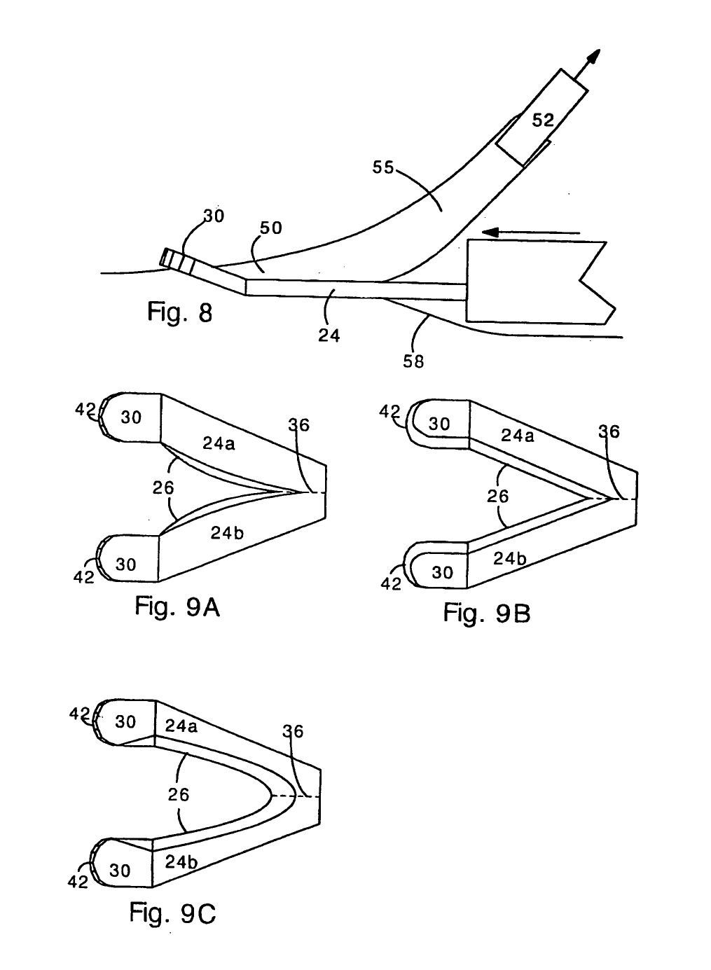

FIG. 8 illustrates the operation of the epithelium cutter after the initial incision of FIG. 7. In FIG. 8, the tweezer tips 52 grasp a strip of removed skin 55 and pull upwardly and to the right so that the removed skin strip 55 is under tension. The V-shaped blade 24 is pushed to the left, so that the skin 50 is separated from underlying vascular tissue 58. The present epithelium cutter can remove an arbitrarily long section of skin by the technique shown in FIG. 8. The upwardly angled end portions 30 help to prevent the V-shaped blade 24 from digging into the vascular tissue and cutting too deeply. The upwardly angled end portions 30 also help to stabilize the cutting action, so that the depth and location of the incision can be easily and accurately controlled by the surgeon. Further, the upwardly angled end portions 30 prevent the V-shaped blade 24 from digging into the vascular tissue.

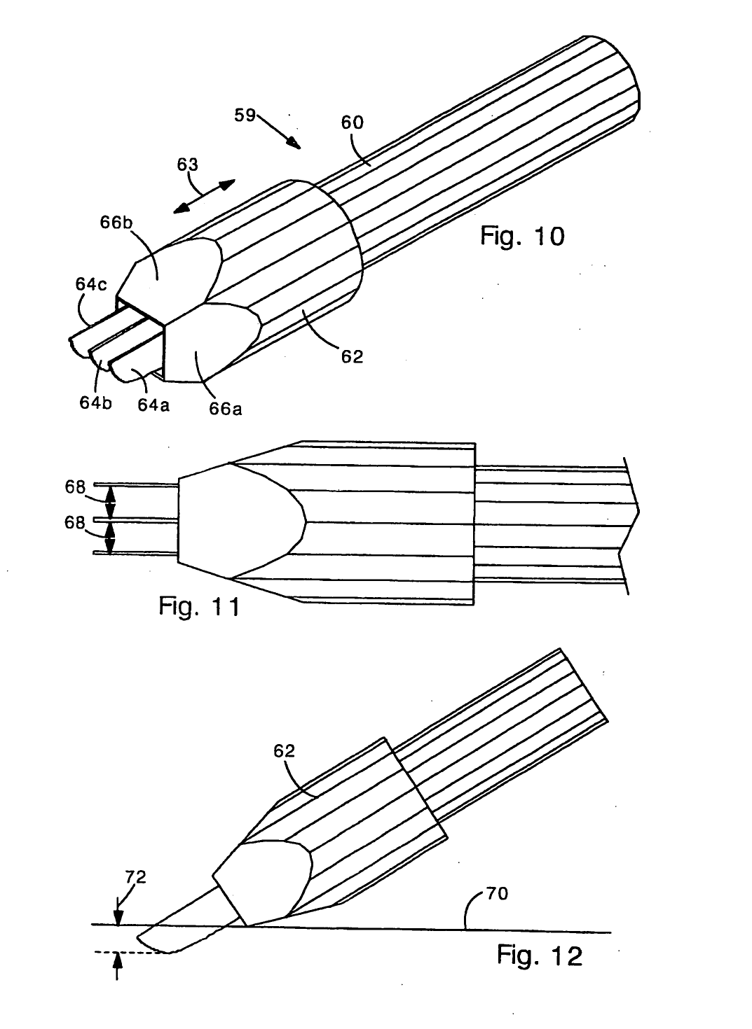

FIGS. 9A-9C illustrate alternative embodiments of the V-shaped blade. It should be understood from these figures that the “V” shape can encompass “U” shapes, or possibly even semicircular shapes. What is requires is that the epithelium is collected within a confined area and cut by the blade edges.

FIG. 9A: The sharp inner edges 26 have a convex shape. The end portions 30 do not have any sharp cutting edges.

FIG. 9B: The outside surfaces 42 are sharp and provide cutting action.

FIG. 9C: The sharp inner edges 26 have a concave shape.

The present invention may also include a rake cutter for use in combination with the present epithelium cutter (although it will be clear to those skilled in the art that the epithelium cutter might be used without the rake cutter). An exemplary rake cutter 59 is shown in FIG. 10. The rake cutter 59 comprises a handle 60, a movable sheath 62, and three parallel, spaced apart cutting blades 64a 64b 64c. The blades 64a 64b 64c are attached to the end of the handle 60. The sheath is movable along the handle 60 in direction 63. The blades 64 can be covered or uncovered by the sheath 62 by moving the sheath along the handle.

Preferably, the sheath comprises beveled surfaces 66a 66b. The beveled surfaces can be placed against the skin during cutting so that the rake cutter is stabilized (e.g., so that the blades are maintained perpendicular to the skin during cutting). The rake cutter sheath 62 can have 3, 4 or more beveled surfaces 66. Each beveled surface 66 can have a different angle. Preferably, the beveled surfaces of the sheath are aligned with the blades so that the blades are perpendicular to the skin surface when one of the beveled surfaces 66 is placed against the skin surface.

FIG. 11 shows a top view of the rake cutter. The blades 64 are preferably equally spaced as shown. The spacing 68 between the blades 64 can be in the range of about ⅛-1 inch, or, more typically, in the range of about ¼-½ inch. Preferably, in one embodiment of the invention, the spacing 68 between the blades 64 is equal to or slightly less than or slightly more than the cutting width 40 of the epithelium cutter (see FIG. 3).

FIG. 12 shows a side view of the operation of the rake cutter. In operation, the position of the sheath 62 is adjusted for a desired cutting depth. The blades 64 are pushed into or against a skin surface 70 until a beveled surface is in contact with the skin. Each of the blades 64 will produce a cut having a constant and accurate depth 72.

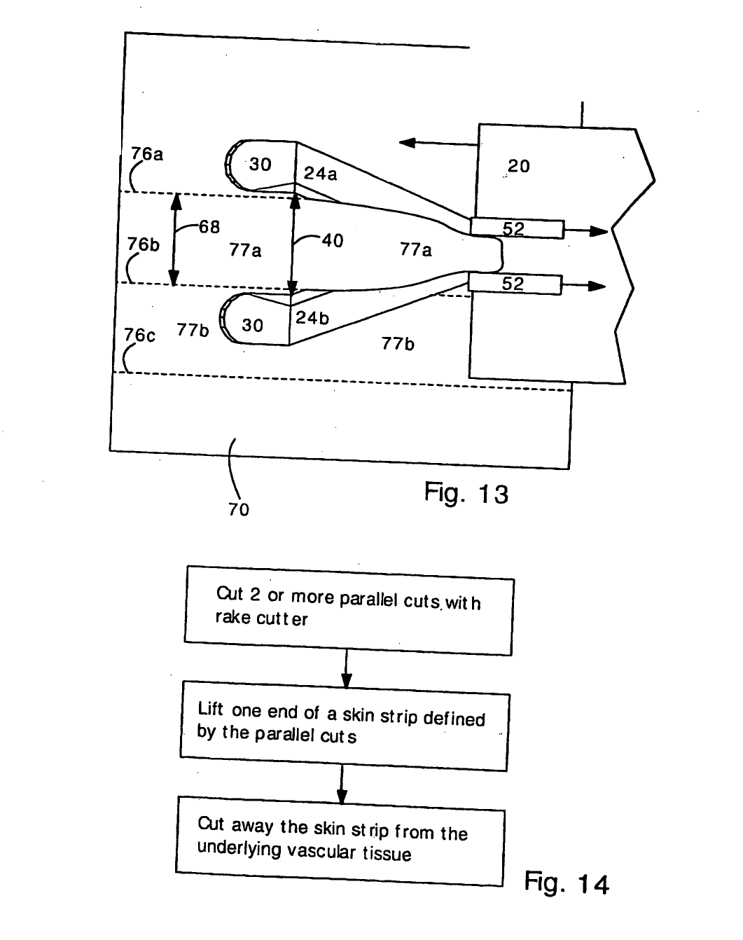

FIG. 13 shows a top view of the epithelium cutter illustrating how the epithelium cutter is used in combination with the rake cutter. The skin surface 70 has three parallel cuts 76a 76b 76c made by the rake cutter 59. The three parallel cuts 76 define two adjacent skin strips 77a 77b that remain attached to underlying vascular tissue. To remove the skin strip 77a the V-shaped blade 24 is moved under the skin strip 77a so that it is cut away from the vascular tissue. Tweezer tips 52 grasp the skin strip 77a and apply tension as it is cut. In order to remove skin from an entire area, many parallel, adjacent skin strips can be removed.

FIG. 14 shows a flow chart describing a method for skin removal according to the present invention. Preferably, the cutting width 40 of the V-shaped blade 24 is equal to or slightly greater (e.g. 5-25% greater) than the blade spacing 68 and distance between the cuts 76. An adequate cutting width 40 ensures that the skin strip 77a is completely removed.

The cuts 76 made by the rake cutter make it unnecessary for the V-shaped blade 24 to cut through the skin (except at the start of cutting). The V-shaped blade 24 only needs to cut through underlying tissue, which makes the procedure faster and more controllable.

In the preferred commercial embodiment, the epithelium cutter will be a single use product, as will the rake blade cutter. The V-shaped blade of the epithelium cutter will be insert molded into a plastic handpiece, and it will preferably be packed and sold sterile. The rake blade cutter might also be packed together with the epithelium cutter, or, alternatively, it can be separately packed and sold separately.

It will be clear to one skilled in the art that the above embodiment may be altered in many ways without departing from the scope of the invention. Accordingly, the scope of the invention should be determined by the following claims and their legal equivalents.

What is claimed is:

1. An epithelium cutter for removing epithelium, comprising:
a) a handle for grasping;
b) a V-shaped blade attached to an end of the handle, wherein the V-shaped blade has sharp inner edges facing away from the handle.

2. The epithelium cutter of claim 1, wherein the handle has a curved shape extending in an upward, vertical direction.

3. The epithelium cutter of claim 2, wherein the V-shaped blade is disposed in a horizontal plane.

4. The epithelium cutter of claim 1, further comprising a head portion, wherein the head portion is substantially planar or parallel with the V-shaped blade.

5. The epithelium cutter of claim 1, wherein the V-shaped blade has upwardly angled end portions.

6. The epithelium cutter of claim 5, wherein the end portions have outside surfaces that are not sharp and do not provide cutting action.

7. The epithelium cutter of claim 1, wherein the V-shaped blade comprises two separately fabricated blades butted, welded, or soldered together.

8. The epithelium cutter of claim 1, wherein the V-shaped blade is attached to the handle by one or more set screws.

9. A rake cutter for creating parallel cuts, comprising:
a) a handle for grasping;
b) a plurality of spaced apart, parallel blades extending from an end of the handle;
c) a sheath covering the blades, wherein the sheath is movable along the handle so as to expose an adjustable length of the blades.

10. The rake cutter of claim 9, wherein the exposed length of the blades is equal.

11. The rake cutter of claim 9, wherein the sheath has at least one beveled surface.

12. The rake cutter of claim 9, wherein the sheath can be locked in position.

13. A kit for epithelium removal, comprising:
an epithelium cutter, comprising:
a) a handle for grasping;
b) a V-shaped blade attached to an end of the handle, wherein the V-shaped blade has sharp inner edges facing away from the handle, and wherein the V-shaped blade has a cutting width; and
a rake cutter for creating parallel cuts, comprising:
a) a handle for grasping;
b) a plurality of spaced apart, parallel blades extending from an end of the handle; and
c) a sheath covering the blades, wherein the sheath is movable along the handle so as to expose an adjustable length of the blades, and wherein a distance between adjacent blades is equal to or slightly less than the cutting width of the V-shaped blade.

14. The kit of claim 13, wherein the distance between adjacent blades is equal to or up to 20% more or less than the cutting width.

15. A method for removing epithelium, comprising the steps of:
a) lifting a portion of skin to form a skin mound;
b) pushing a V-shaped blade having sharp inner edges into the skin mound, such that the sharp inner edges cut away the skin from underlying vascular tissue.

16. The method for removing epithelium of claim 15, wherein the skin is held in tension during step (b).

17. The method for removing epithelium of claim 15, further comprising the step of creating two parallel cuts in a skin surface prior to step (a), thereby defining a skin strip, wherein one end of the skin strip is lifted in step (b), and wherein the V-shaped blade cuts away the skin strip from underlying vascular tissue.
